(19) United States
(12) Patent Application Publication
(10) Pub. No.: US 2006/0019951 A1
(43) Pub. Date: Jan. 26, 2006

(54) Vacuolins

(75) Inventors: Tomas Kirchhausen, Brighton, MA (US); Jan Cerny, Jamaica Plain, MA (US)

Correspondence Address: LAHIVE & COCKFIELD, LLP., 28 STATE STREET, BOSTON, MA, 02109, US

(73) Assignee: CBR Institute for Biomedical Research, Boston, MA (US)

(21) Appl. No.: 11/021,840

(22) Filed: Dec. 23, 2004

Related U.S. Application Data
(63) Continuation of application No. PCT/US03/20270, filed on Jun. 25, 2003.
(60) Provisional application No. 60/391,331, filed on Jun. 25, 2002.

(51) Int. Cl. A61K 31/5377; C07D 413/04

(52) U.S. Cl. 514/235.5; 544/106; 544/112

(57) ABSTRACT

The present invention provides compositions and methods relating to vacuolins and their uses. Vacuolins are small molecule agents that affect certain membrane fusion events involving intracellular compartments. The invention further provides compositions and methods for altering antigen presentation mediated by class II MHC molecules, and/or for inhibiting histamine release from mast cells.

### RELATED APPLICATIONS

This application is a continuation of PCT/US2003/020270, entitled “Vacuolins,” filed on Jun. 25, 2003, which claims the benefit of U.S. Ser. No. 60/391,331, entitiled “Vacuolins,” filed on Jun. 25, 2002. The entire contents of these applications are hereby incorporated herein by reference.

### BACKGROUND

The formation and trafficking of intracellular vesicles are critical to a variety of biological pathways and events, including protein processing and transport. There is a need for improved understanding of vesicle processing, and for the identification of reagents and methods that can assist in its analysis. There is a further need for the identification of agents that can modulate or regulate intracellular vesicle activities.

### SUMMARY

The present invention provides vacuolin compounds, e.g., isolated vacuolin compounds, compositions comprising them, and related methods. In particular, the invention demonstrates immunologically relevant activities of vacuolins, and provides techniques and reagents for using vacuolins to modulate immunological events or reactions.

In one aspect, the invention provides a composition comprising a vacuolin having the following structure:

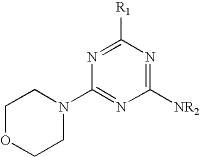

wherein R1 is an amino cycloaliphatic or cycloheteroalkyl group.

In one embodiment, R1 is an aminoaryl group. In another embodiment, R1 is selected from the group consisting of a morpholino, a phenyl amine, a diphenylamine, a substituted phenyl amine, a substituted diphenyl amine, a benzylamine, and a substituted benzylamine. The substituted phenylamine may be a halophenylamine selected from the group consisting of a chlorophenylamine, bromophenylamine, fluorophenylamine, and a iodophenyl amine. In yet another embodiment, the phenylamine has a para, ortho or meta substitution.

In another embodiment, R2 is an arylakyleneamine group. In still another embodiment, R2 is an arylmethyleneamine, an arylethyleneamine, or an arylpropyleneamine group. In another embodiment, R2 is a phenylalkyleneamine group. R2 may also be, for example, a benzylideneamine group or a benzene. In still another embodiment, R2 is substituted with a halogen selected from the group consisting of fluorine, bromine, chlorine, and iodine. In a further embodiment, R2 has a meta, para or ortho substitution.

In another aspect, the invention provides compositions comprising a vacuolin selected from the group consisting of at least one of the vacuolin structures shown in FIG. 1.

In yet another aspect, the invention provides method for modulating vacuolarization in a cell comprising: providing a cell; and contacting said cell with an effective amount of a vacuolin compound, thereby modulating vacuolarization in a cell. In one embodiment, vacuolarization in increased. The method may be carried out in vitro or in vivo.

In still another aspect, the invention provides methods of inhibiting antigen presentation mediated by MHC class II molecules, comprising providing a population of target cells containing internal MHC class II molecules; and contacting the target cells with an effective amount of a vacuolin, so that, if the cells were triggered to externalize the MHC class II molecules, such externalization would be reduced as compared with externalization by comparable cells not so contacted.

Another aspect of the invention provides methods of inhibiting externalization of intracellular compartments, the method comprising steps of providing a population of target cells containing intracellular compartments susceptible of externalization; and contacting the target cells with an effective amount of a vacuolin compound so that, if the contacted cells were triggered to externalize the intracellular compartments, a lower level of externalization would be observed than is seen with comparable cells not so contacted. In one embodiment, the intracellular compartments contain MHC class II molecules. In another embodiment, the intracellular compartments contain histamine. In yet another embodiment, the MHC class II molecules contain antigens that induce IgG switching. In still another embodiment, the step of providing comprises providing a population of graft cells containing MHC class II molecules that carry host protein fragments as antigens for presentation. In another embodiment, the step of providing comprises providing gut cells and the MHC class II molecules carry gluten.

In another aspect, the invention provides methods of inhibiting trafficking of intracellular compartments in a cell, the method comprising steps of providing a cell; and contacting the cell with an effective amount of a vacuolin, thereby inhibiting trafficking of intracellular compartments. The method may be carried out in vitro or in vivo.

Another aspect of the invention provides methods of treating an intracellular trafficking-related disease or disorder in a subject, comprising administering to a subject an effective amount of a vacuolin of the invention.

### BRIEF DESCRIPTION OF DRAWINGS

FIG. 1 shows the structures of certain preferred vacuolins.

FIG. 2 highlights a structural domain conserved among 4 of the 6 active compounds depicted in FIG. 1.

FIGS. 3-5 depict the visualization of a variety of intracellular markers revealing that voids formed within cells exposed to vacuolins were derived from late endosomes, multi-vesicular compartments, and lysosomes.

FIG. 6 depicts the formation of vacuoles.

FIGS. 7A-7B depict the formation of vacuoles.

FIGS. 8-10 depict results of experiments directed at identifying conditions under which vacuole formation is blocked in cells exposed to a vacuolin.

FIG. 11 illustrates that vacuoles are likely to accumulate Cl ions.

FIG. 12 depicts the reversibility of vacuole formation in cells contacted with the indicated vacuolin.

FIG. 13 depicts results of experiments indicating that 19m6 vacuolin is cytostatic, but not cytotoxic.

FIG. 14 depicts vacuolation of the MHCII/GFP compartment in bone marrow derived dendritic cells from MHCII/GFP-Kl mouse which were contacted with various vacuolins.

FIG. 15 presents representative data demonstrating that inventive vacuolins can block the LPS-stimulated surface expression of MHC class II molecules.

FIG. 16, panels A and B, show that inventive vacuolin compounds block trafficking and tubulation of intracellular compartments containing MHC class II molecules in GFP-KI murine dendritic cells.

FIG. 17 depicts results of experiments indicating that inventive vacuolin compounds can block the appearance of lysosomal markers on the cell surface after scraping.

### DEFINITIONS

Small Molecule: As used herein, the term small molecule refers to a chemical compound, whether naturally-occurring or artificially created (e.g., via chemical synthesis) that has a relatively low molecular weight, preferably less than about 1500. In certain embodiments, a small molecule is characterized in that it contains several carbon-carbon bonds; often, a small molecule will be characterized by having a molecular weight less than about 1000, preferably less than about 500.

Vacuolin: The present invention defines a class of compounds that, when administered to cells as described herein, results in vacuolarization of those cells. Such compounds are vacuolins as that term is used herein.

### DESCRIPTION OF CERTAIN PREFERRED EMBODIMENTS

Vacuolins

As described herein, the present invention identifies a class of compounds that, when contacted with cells, results in vacuolarization of those cells (see, for example, Example 1).

Preferred vacuolins have a chemical structure depicted below:

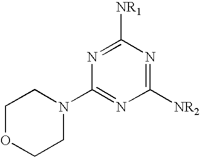

In certain preferred embodiments, R₁ comprises a benzene ring, optionally substituted with at least one halogen. In other preferred embodiments, R₂ comprises a benzene ring, optionally attached via a short alkyl linker.

Particularly preferred vacuolins have the following structure:

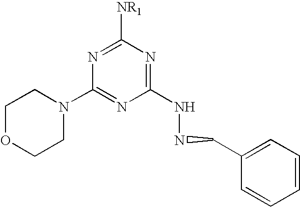

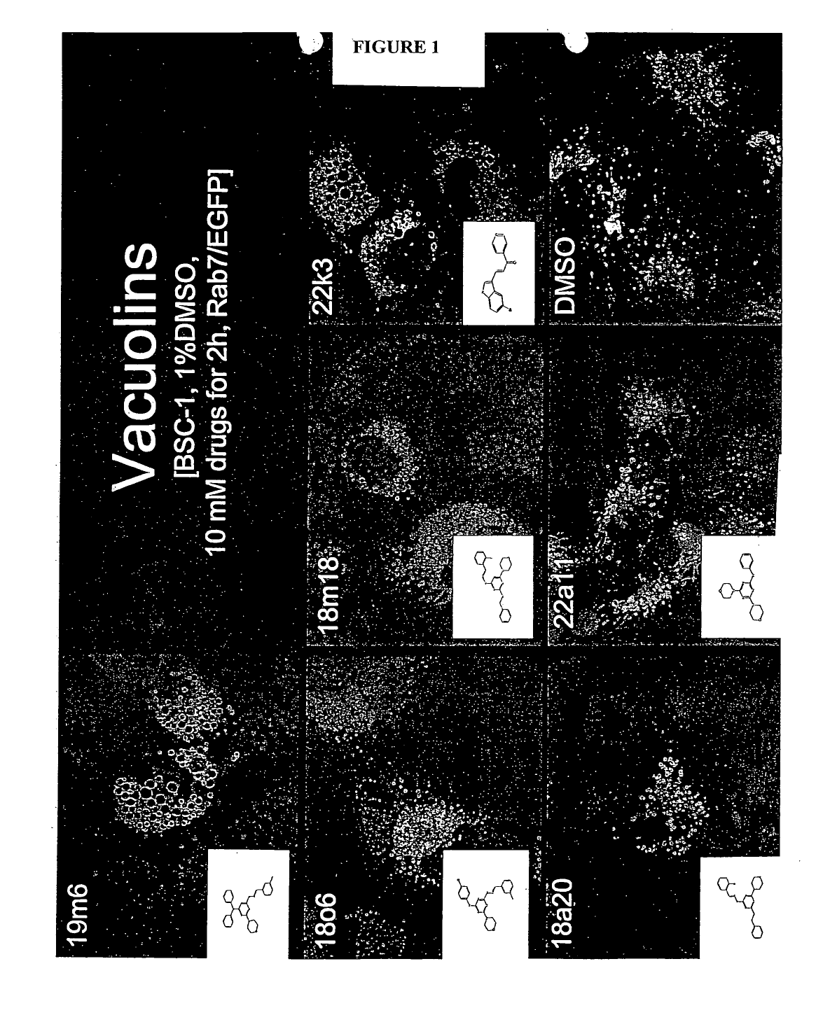

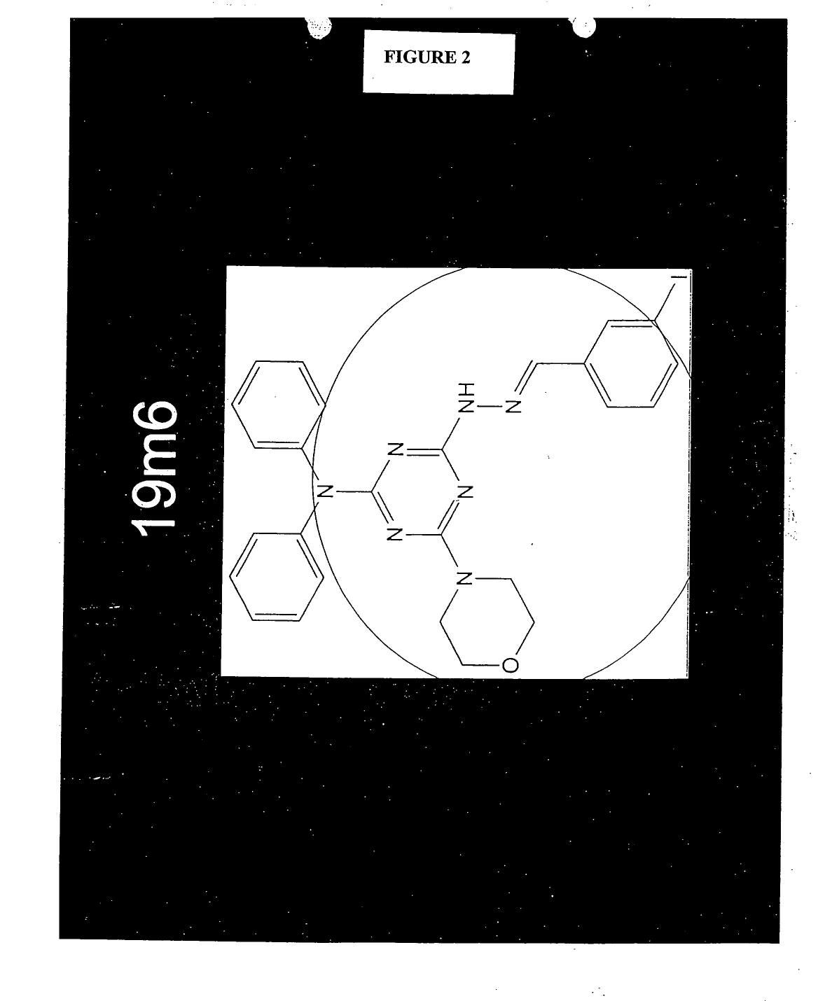

Certain preferred vacuolins are the compounds whose structures are shown in FIG. 1.

Those of ordinary skill in the art will appreciate that vacuolin compounds of the present invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. It is to be understood that the invention encompasses every possible isomer such as geometric isomer, optical isomer, stereoisomer and tautomer based on asymmetric carbon, which can occur in the structures of the inventive compounds, and mixtures of such isomers and compositions comprising those compounds, and is not limited to the specific stereochemistry shown for the compounds disclosed in the present specification. It will be further appreciated that the absolute stereochemistry of some of the compounds recited in the Exemplification herein may not have been determined, and that when a stereochemistry was assigned for those compounds it is meant to be tentative and to indicate that a set of diastereomers exists for those compounds and/or that a diastereomer was isolated in pure form. Furthermore, it will be appreciated that certain of the compounds disclosed herein contain one or more double bonds and these double bonds can be either Z or E, unless otherwise indicated. In certain embodiments, the compounds of the invention are enantiopure compounds. In certain other embodiments, a mixture of stereoisomers or diastereomers are provided.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof.

Pharmaceutically acceptable derivatives thus include among others pro-drugs. A pro-drug is a derivative of a compound, usually with significantly reduced pharmacological activity, which contains an additional moiety which is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction.

For example, oxygen, sulfur, nitrogen and carbon protecting groups may be utilized. Certain exemplary oxygen protecting groups include, but are not limited to methyl ethers, substituted methyl ethers (e.g., MOM (methoxymethyl ether), MTM (methylthiomethyl ether), BOM (benzyloxymethyl ether), PMBM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals.

Exemplary nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. As will be appreciated by those of ordinary skill in the art, a variety of additional equivalent protecting groups can be utilized in accordance with the present invention (see, for example, “Protective Groups in Organic Synthesis” Third Ed. Greene, T. W. and Wuts, P. G., Eds., John Wiley & Sons, New York: 1999, the entire contents of which are hereby incorporated by reference).

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms.

Furthermore, this invention is not intended to be limited in any manner by the permissible substituents of organic compounds. Combinations of substituents and variables envisioned by this invention are preferably those that result in the formation of stable compounds useful in blocking externalization of intracellular compartments and/or in vacuolarizing cells. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-l-yl, and the like. Representative alkynyl groups include, but are not limited to, ethynyl, 2-propynyl (propargyl), 1-propynyl and the like.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of sutstituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. It will also be appreciated that aryl and heteroaryl moieties, as defined herein may be attached via an alkyl or heteroalkyl moiety and thus also include -(alkyl)aryl, -(heteroalkyl)aryl, -(heteroalkyl)aryl, and -(heteroalkyl)heteroaryl moieties. Thus, as used herein, the phrases “aryl or heteroaryl” and “aryl, heteroaryl, -(alkyl)aryl, -(heteroalkyl)aryl, -(heteroalkyl)aryl, and -(heteroalkyl)heteroaryl” are interchangeable. Substituents for exemplary aryl and heteroaryl moieties include, but are not limited to, any of the previously mentioned substitutents, i.e., the substituents recited for aliphatic moieties, or for other moieties as disclosed herein, resulting in the formation of a stable compound.

In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bycyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties which contain one or more oxygen sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tr-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substitutents described above and herein may be substituted or unsubstituted. Additional examples or generally applicable substituents are illustrated by the specific embodiments shown in the Examples which are described herein.

Identification of Vacuolins

Example 1 describes the particular assay system used to identify vacuolins as described herein. As indicated in that Example, and as would be readily appreciated by those of ordinary skill in the art, additional or alternative vacuolins could readily be identified by applying the same screen to other chemical compounds.

For instance, extracts containing natural products are often used as sources of test compounds for biological assays (see, for example, Clark, Pharm. Res., 13:1133-1144, 1996, incorporated herein by reference). Alternatively or additionally, synthetic compounds can be utilized. As will be appreciated by those of ordinary skill in the art, the development of combinatorial chemistry and split-pool synthesis techniques have added to the repertoire complex compound libraries, the products of laboratory syntheses, as a source of small molecules to be screened for novel compounds with biological activity (see for example, Tan et al., J. Am. Chem. Soc., 120:8565-8566, 1998, incorporated herein by reference). However, small molecules synthesized by parallel synthesis methods and by traditional methods (one-at-a-time synthesis and modifications of these structures) can also be utilized in the compositions and methods of the present invention, as can naturally occurring compounds, or other collections of compounds.

As will be realized by one of ordinary skill in the art, in split-and-pool techniques (see, for example, Furka et al., Abstr. 14th Int. Congr. Biochem., Prague, Czechoslovakia, 5:47, 1988; Furka et al., Int. J. Pept. Protein Res. 37:487, 1991; Sebestyen et al., Bioorg. Med. Chem. Lett. 3:413, 1993; each of which is incorporated herein by reference), a mixture of related compounds can be made in the same reaction vessel, thus substantially reducing the number of containers required for the synthesis of very large libraries, such as those containing as many as or more than one million library members. As an example, a solid support bound scaffold can be divided into n vessels, where n represents the number of species of reagent A to be reacted with the support bound scaffold. After reaction, the contents from n vessels are combined and then split into m vessels, where m represents the number of species of reagent B to be reacted with the support bound scaffold. This procedure is repeated until the desired number of reagents are reacted with the scaffold structures to yield a desired library of compounds.

As mentioned above, the use of parallel synthesis methods are also applicable. Parallel synthesis techniques traditionally involve the separate assembly of products in their own reaction vessels. For example, a microtiter plate containing n rows and m columns of tiny wells which are capable of holding a small volume of solvent in which the reaction can occur, can be utilized. Thus, n variants of reactant type A can be reacted with m variants of reactant type B to obtain a library of n x m compounds.

Preparation of Vacuolins

Once identified, vacuolins of the present invention may be prepared by any available means. In some cases, the vacuolins may be compounds that occur naturally and therefore may be prepared from a natural source using known purification and isolation technologies. In other cases, the vacuolins may not be naturally-occurring, or may not be readily isolatable from a natural source, and therefore may instead be prepared using synthetic techniques, as is known in the art. Any appropriate synthetic techniques may be employed, including those that utilize only chemical reagents or those that utilize biological reagents such as synthetic enzymes. Alternatively or additionally, synthetic and isolationary techniques may be combined in the preparation of inventive vacuolins. Accordingly, the present invention includes isolated vacuolin compounds. The terms “isolated” or “substantially purified” as used interchangeably herein refer to vacuolin compounds in a non-naturally occurring state. The compounds can be substantially free of cellular material or culture medium when naturally produced, or chemical precursors or other chemicals when chemically synthesized.

Therapeutic Uses

As described herein, inventive vacuolin compounds, and compositions containing them, have unexpected immunological effects. For example, vacuolins can inhibit induced transportation of MHC class II compounds. Inventive vacuolins can therefore be used to regulate or modulate cellular events that rely upon or involve such transportation. Certain such cellular events are mentioned below; others will be apparent to those of ordinary skill in the art.

For example, according to the present invention, vacuolins may be used to regulate or inhibit IgG switching, and therefore to reduce IgG production. Vacuolins may alternatively or additionally inhibit IgG secretion.

Alternatively or additionally, vacuolins may be employed to reduce graft versus host response, for example as often occurs after bone marrow transplantation. Graft versus host response results from production of antigen presenting cells within a graft in which host antigens are presented as foreign. Such undesirable presentation of host antigens often occurs acutely in the gut and the skin. According to certain preferred embodiments of the invention, vacuolins may be applied locally (e.g., topically) to alleviate the graft versus host response.

Alternatively or additionally, inventive vacuolins may be utilized to suppress other undesirable immune reactions that involve MHC class II presentation of antigens. For example, inventive vacuolins could be utilized to inhibit an undesirable immune response in the gut to ingested gluten.

As also described herein, vacuolin compounds, and compositions containing them, can derail cellular secretion processes. Accordingly, vacuolins may be utilized to inhibit processes that require or involve transport of intracellular vesicles, or components thereof.

To give but one example, vacuolins may be utilized to inhibit release of histamine-containing vesicles from mast cells. Such inhibition could be useful, for example, in the treatment or prevention of allergic or asthmatic reactions, including anaphylactic reactions, such as can occur in sensitive individuals exposed to drugs, asthamtic triggers, allergens (e.g., hay fever allergens, ivies, insect stings, etc.).

Accordingly, in one embodiment, vacuolins may be used to treat or prevent intracellular trafficking-related diseases or disorders. As used herein, the term “intracellular trafficking-related disease or disorder” includes any disease or disorder which is caused by or related to transport or trafficking of intracellular vesicles or compartments, e.g., the externalization of intracellular compartments, or the transport of intracellular proteins within intracellular compartments. For example, intracellular trafficking-related diseases or disorders include inflammatory or immune diseases or disorders, including, without limitation, viral infection, inflammatory bowel disease, ulcerative colitis, Crohn's disease, leukocyte adhesion deficiency II syndrome, peritonitis, chronic obstructive pulmonary disease, lung inflammation, asthma, graft versus host response, acute appendicitis, septic shock, nephritis, amyloidosis, rheumatoid arthritis, chronic bronchitis, sarcoidosis, scleroderma, lupus, polymyositis, Reiter's syndrome, psoriasis, pelvic inflammatory disease, inflammatory breast disease, orbital inflammatory disease, immune deficiency disorders, and autoimmune disorders. Accordingly, the vacuolins of the invention may be used to treat or prevent inflammatory or immune diseases or disorders in a subject.

Research Uses

In addition to the various pharmaceutical uses described above, vacuolin compounds of the present invention have utility in a variety of research applications, e.g., in vitro assays, including, for example, as chemical probes for analyzing intracellular trafficking and/or membrane fusion events. For example, the vacuolins of the invention may be used for analyzing the kinetics of intracellular trafficking, antigen presentation, histamine release, and/or membrane fusion events, in assays for identification of compounds which modulate these processes, or in other in vitro assays. Those of ordinary skill in the art will appreciate that the field of chemical genetics attempts to identify chemical agents with definable effects on biological events, pathways, or products so that these agents may be used as tools to analyze the relevant biological events, pathways, or products. Vacuolins of the present invention are particularly well suited for such studies. Accordingly, the present invention also includes assays, e.g., in vitro assays, utilizing the vacuolins of the present invention to analyze vacuolarization, intracellular trafficking, antigen presentation, membrane fusion events, and related cellular processes. Furthermore, the vacuolins of the present invention may also be used in screening assays to identify second generation vacuolins, e.g., molecules having modified chemical structures which function as vacuolins.

Formulations

As described herein, inventive vacuolin compounds may be utilized in any of a variety of contexts, and may be formulated appropriately according to known principles and technologies.

For example, vacuolins may be provided in substantially pure form, typically in a standard organic solvent such as DMSO. Alternatively or additionally, vacuolins may be formulated as a pharmaceutical composition, for example being combined with a pharmaceutically acceptable carrier. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals with little or no undue toxicity, irritation, allergic response and the like, and are commensurate with a reasonable benefit/risk ratio. Pharmaceutically acceptable salts of amines, carboxylic acids, and other types of compounds, are well known in the art (see, for example, Berge, et al. J. Pharmaceutical Sciences, 66:1-19, 1977, incorporated herein by reference).

The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Inventive vacuolins may be formulated or administered together with any other known agent having a complementary biological effect. For example, vacuolins may be combined with steroids or other immunomodulating agents in order to regulate immunological events as described herein.

It will be appreciated that the compounds and compositions, according to the method of the present invention, may be administered using any effective amount and any effective route of administration. Thus, the expression “effective amount” as used herein, refers to a sufficient amount of agent to result in vacuolarization and/or inhibition of compartment trafficking as described herein. The exact amount required may vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment.

The specific therapeutically effective dose level for any particular patient or organism may depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In other embodiments of the invention, vacuolin compounds, or compositions containing them are packages into a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

This invention is further illustrated by the following Exemplification which should not be construed as limiting. The contents of all references, Figures, and published patent applications cited throughout this application are hereby incorporated by reference.

### EXEMPLIFICATION

### Example 1

### Identification of Vacuolins

Vacuolins were first identified in a screen for compounds that inhibit exocytosis. The assay utilized a strain of mammalian cells that expresses the temperature sensitive viral membrane glycoprotein VSVGts045 fused to EGFP (VSVGts045-EGFP). When cells are maintained at the nonpermissive temperature (e.g., 40° C.), the fusion protein is produced but is not released from the endoplasmic reticulum (ER). Shifting the cells to the permissive temperature (e.g., 32° C.) results in release of the fusion protein, and its trafficking to the Golgi apparatus and then to the plasma membrane.

The assay was performed by maintaining cells at the nonpermissive temperature overnight, to allow the fusion protein to be made and to accumulate in the ER. Cells were then exposed to potential chemical agents for a predetermined period of time (e.g., about 30 min) prior to being transferred to the permissive temperature. Treated cells were maintained at the permissive temperature for a predetermined period of time (e.g., 1-2 hours), and then were fixed, analyzed by fluoresence light microscope, and scored for perturbations in the intracellular localization of the VSVGts045-EGFP fusion protein.

The assay was performed with a 10,000-member subset of the Diverset E Chembridge library, with each compound being employed at a nominal final concentration of 100 nM in 1% DMSO. Those of ordinary skill in the art will readily appreciate that any available chemical compounds could be tested in the same way, and that different concentrations could be used.

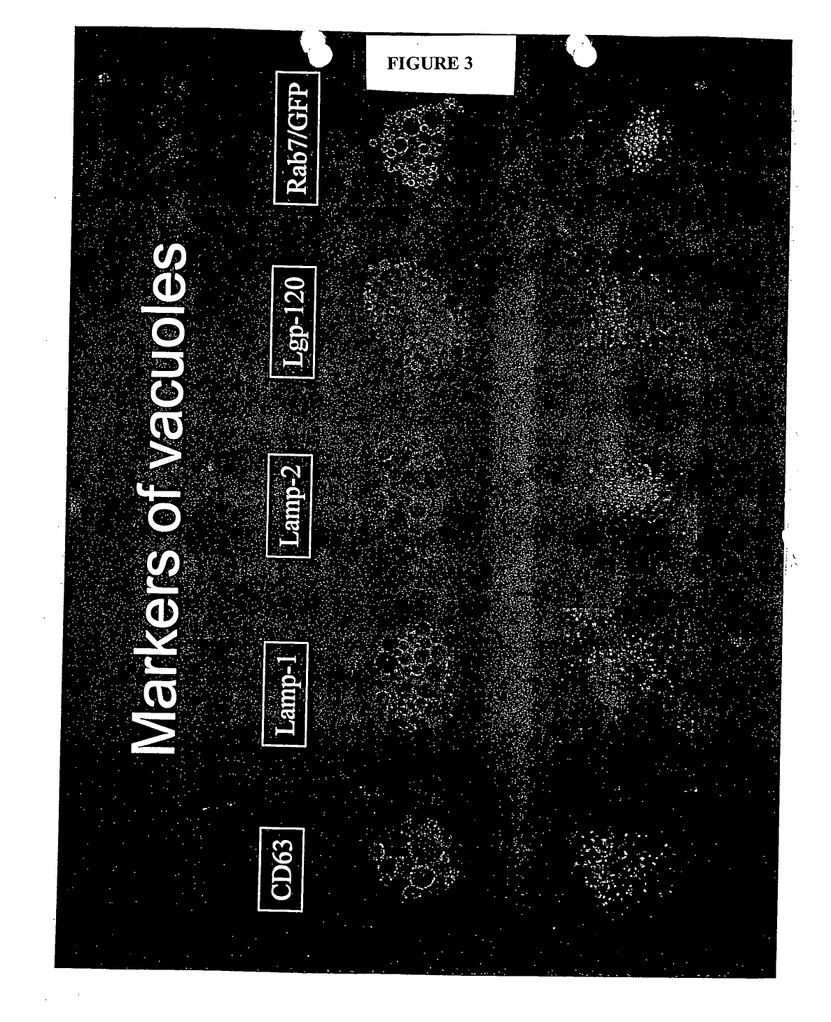

FIG. 1 shows some representative images of a remarkable phenotype observed when certain compounds were tested in this assay. As can be seen, exposure to these compounds resulted in the formation of circular voids, lacking VSVGts045-EGFR, within the cytosol. As can be further seen, several of these compounds are structurally related to one another, and can be described by the general chemical formula: 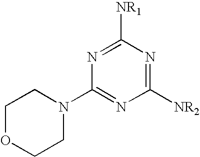
FIG. 2 highlights a structural domain conserved among 4 of the 6 active compounds depicted in FIG. 1.

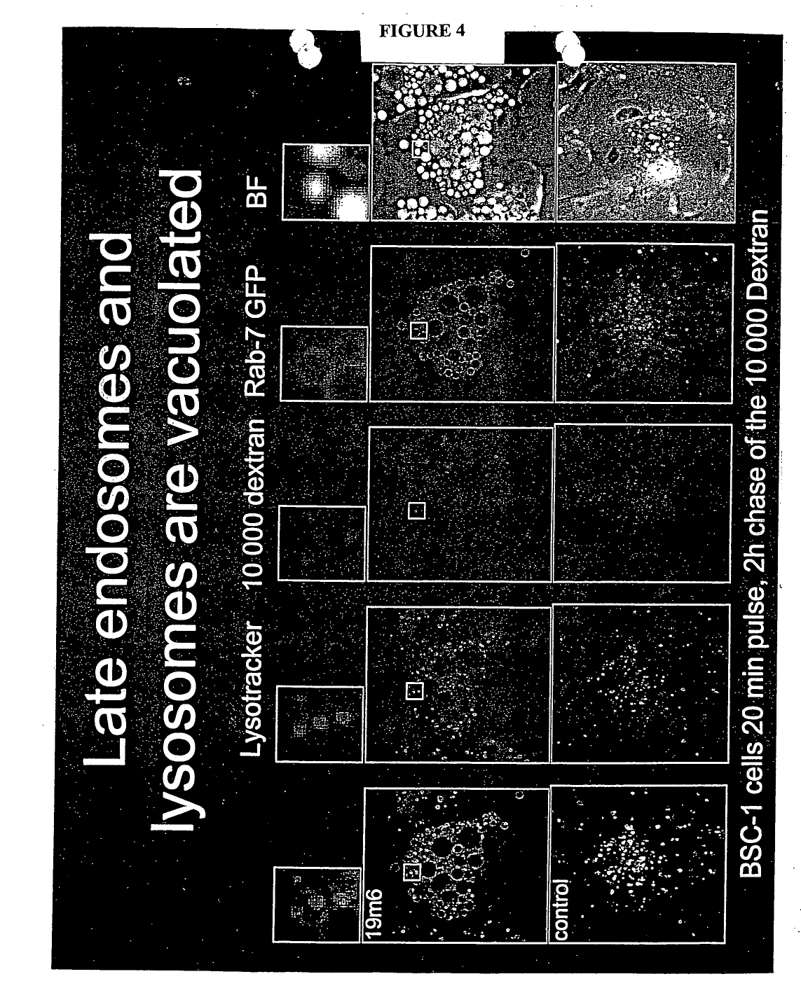

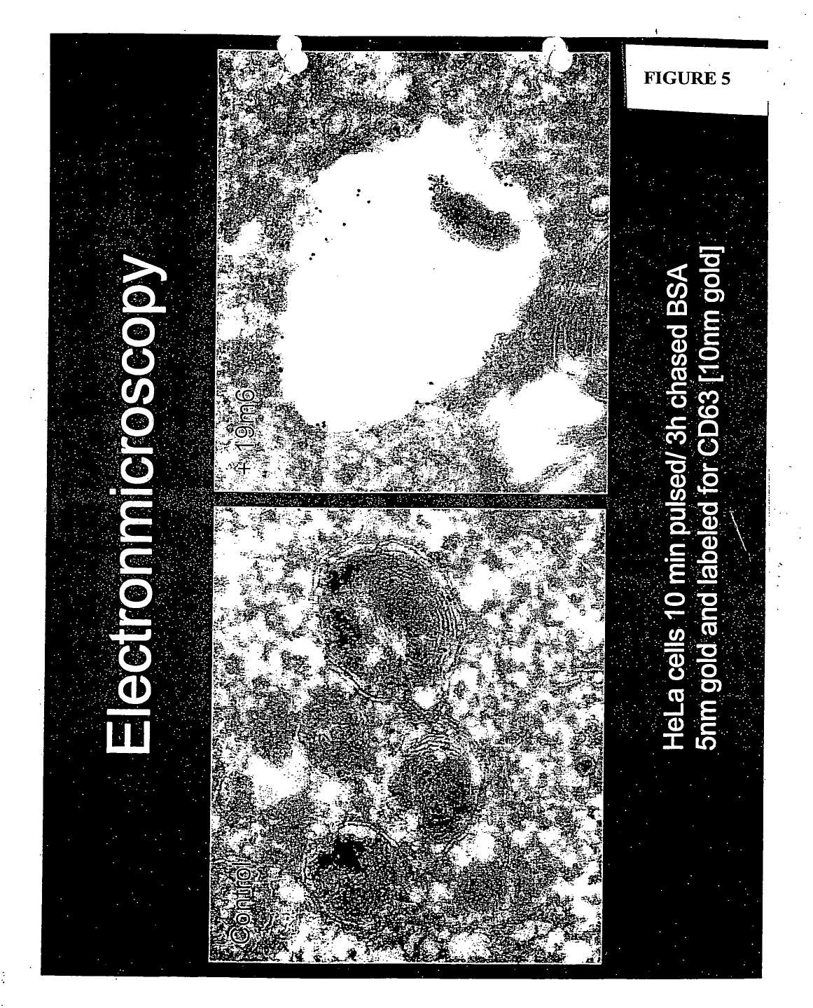

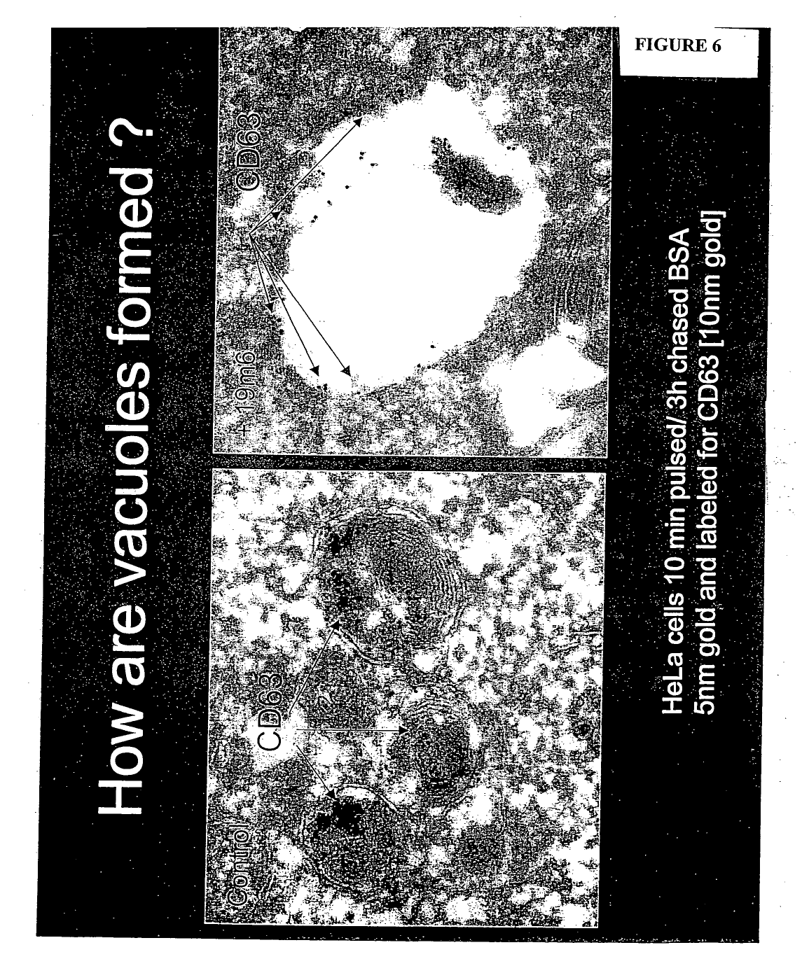

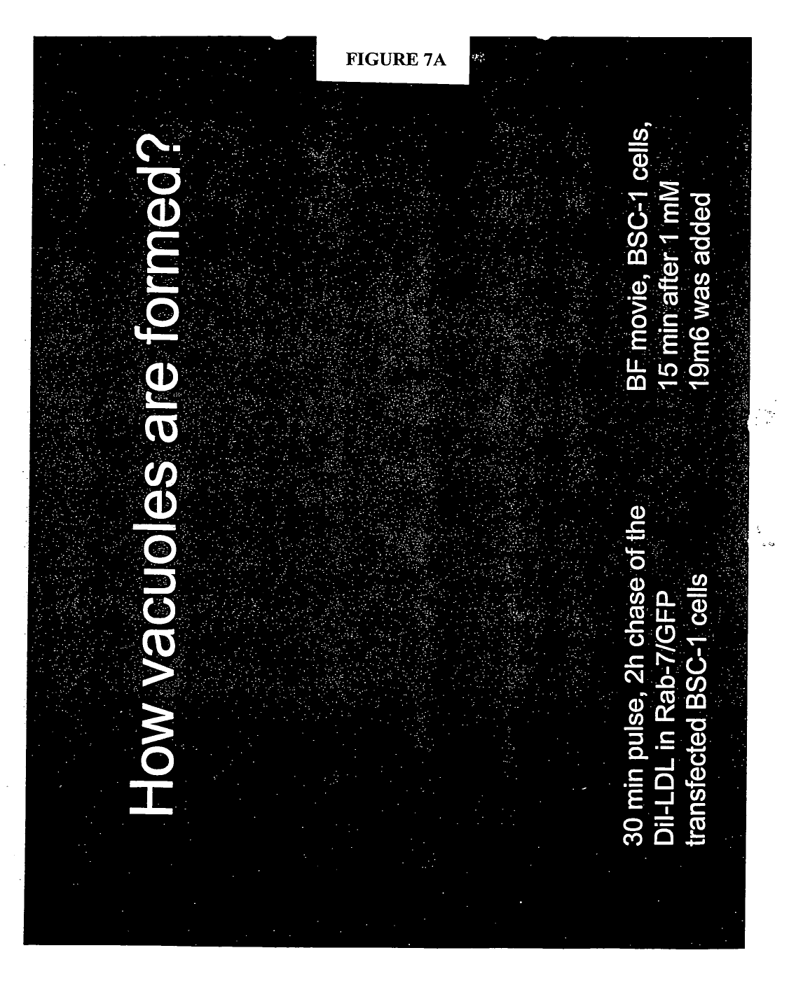

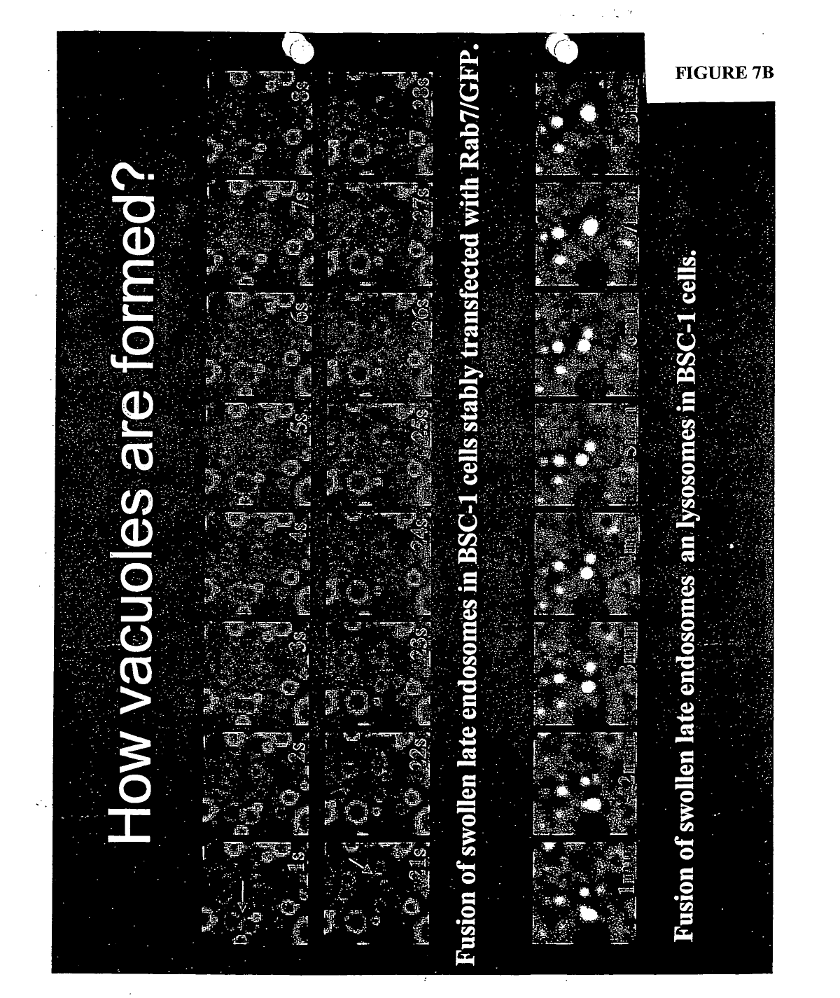

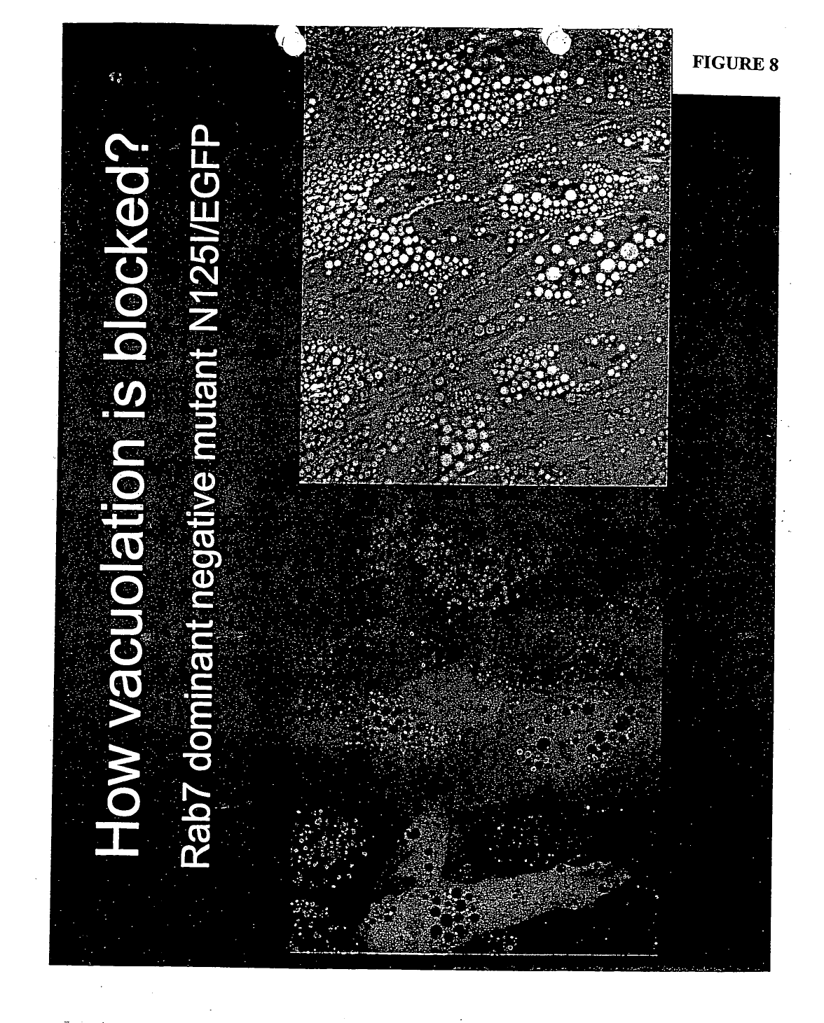

Further studies were performed to evaluate the nature of the observed circular voids. In particular, as shown in FIGS. 3-5, a variety of intracellular markers (e.g., rab7, Ig120, lampI, lampII, lysobisphospatidic acid, CD63, MHC class II, BSA conjugated to colloidal gold) were visualized via light or electron microscopy. These studies revealed that the voids were vacuoles that derived from late endosomes, multi-vesicular compartments, and lysosomes (see, for example, FIGS. 7A-7B). In particular, it was determined that the inner membranes of these intracellular compartments had disappeared and had fused with the limiting membranes. The vacuoles also could fuse with one another. Furthermore, the vacuoles could receive endocytosed material (demonstrated with, e.g., transferrin; LDL-Dil; viruses such as SV40, HIV, etc.; fixable fluorescent dextran). As a result of these studies, the collection of compounds that triggers the observed phenotypic effect was termed the “vacuolins”.

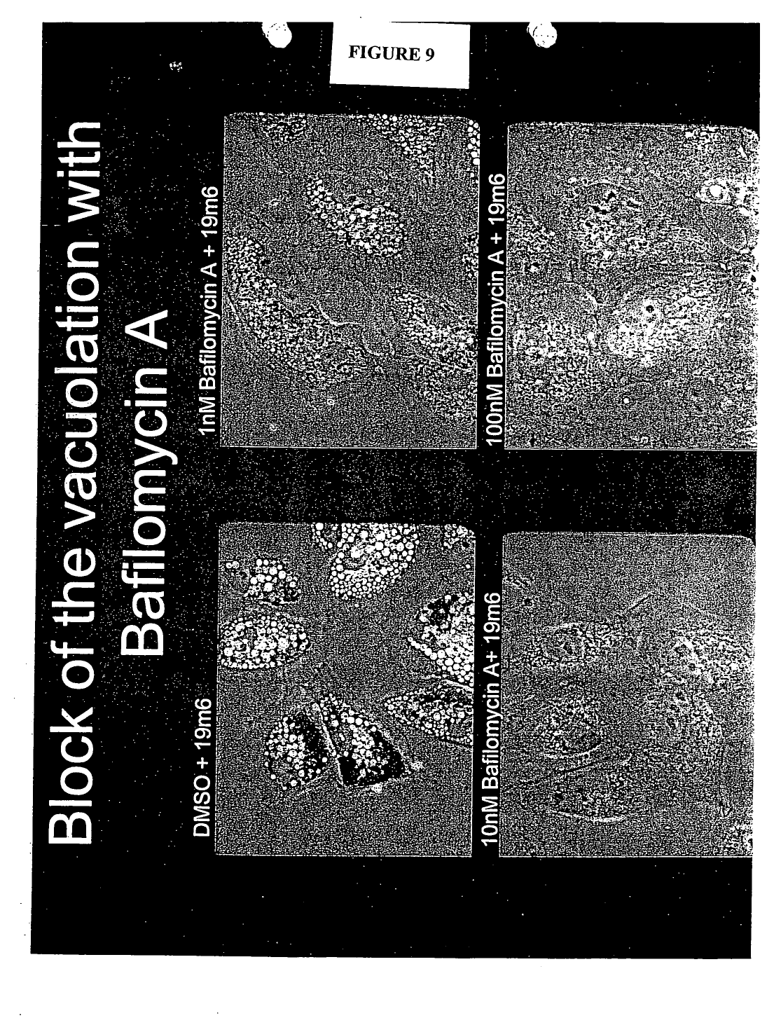

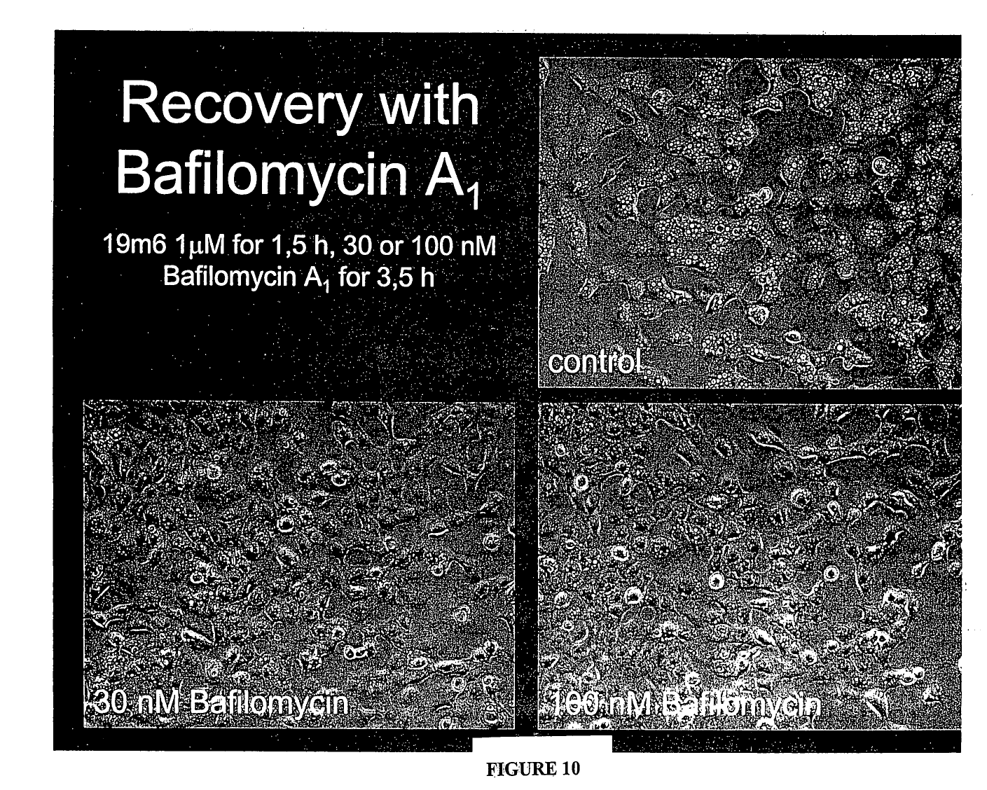

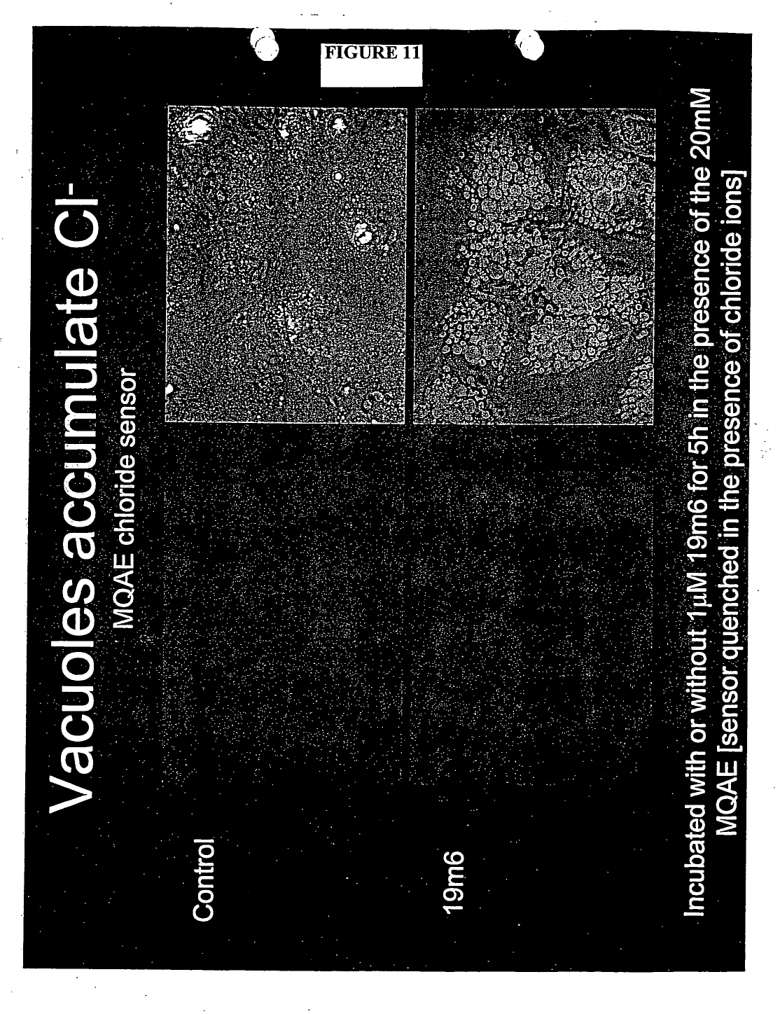

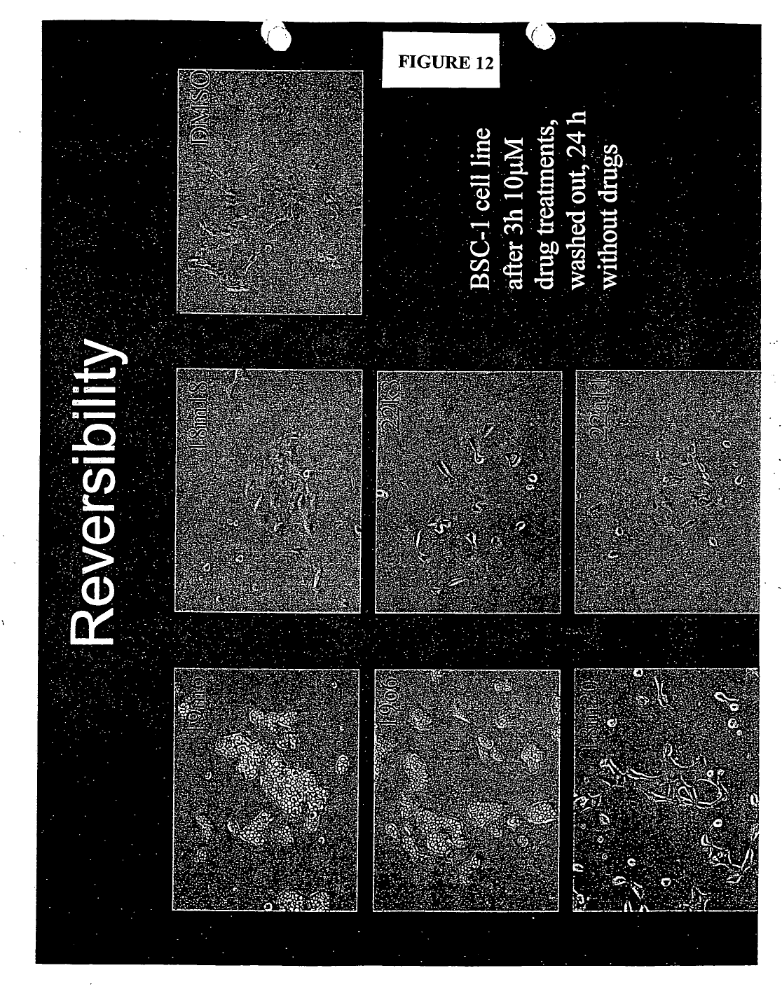

Additional studies were directed at identifying conditions under which vacuole formation is blocked (see, for example, FIGS. 8-10). As can be seen, vacuole formation is inhibited by bafilomycin A, a Na/H pump. It is likely that the intravacuolar pH is higher than the low pH of lysosomes; the vacuoles are likely to accumulate Cl ions (see, for example, FIG. 11).

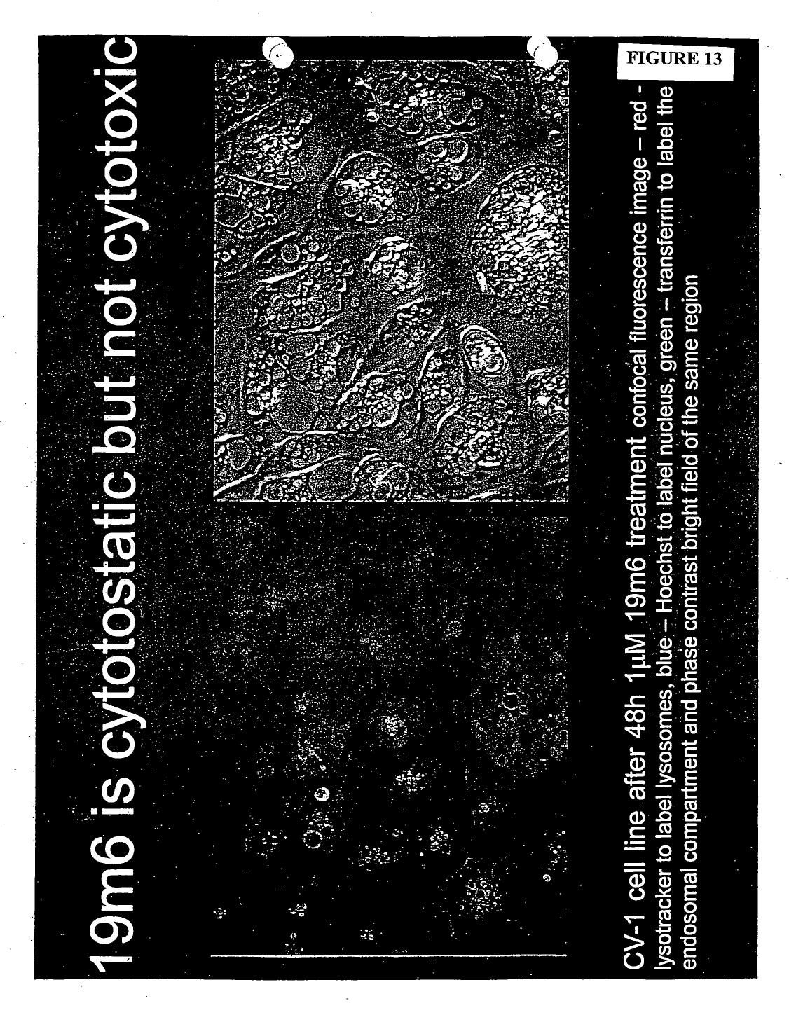

A variety of different cell types, including fibroblast lines, macrophages, dendritic cells, and epithelial cells, were tested for susceptibility to vacuole formation; all vacuolated when contacted with the compounds described herein. With some compounds, the effect was reversible; with others, it was not (see, for example, FIG. 12).

### Example 2

### Cytostatic Effects of Vacuolins

At least some of the vacuolin compounds described herein have cytostatic effects on cells. These compounds do not, however, have obvious effects on intracellular structures or organelles other than their effects on vacuole formation. The compounds do not affect endocytosis from the plasma membrane, or on the organization of the actin and tubulin cytoskeleton. The compounds do not induce apoptosis.

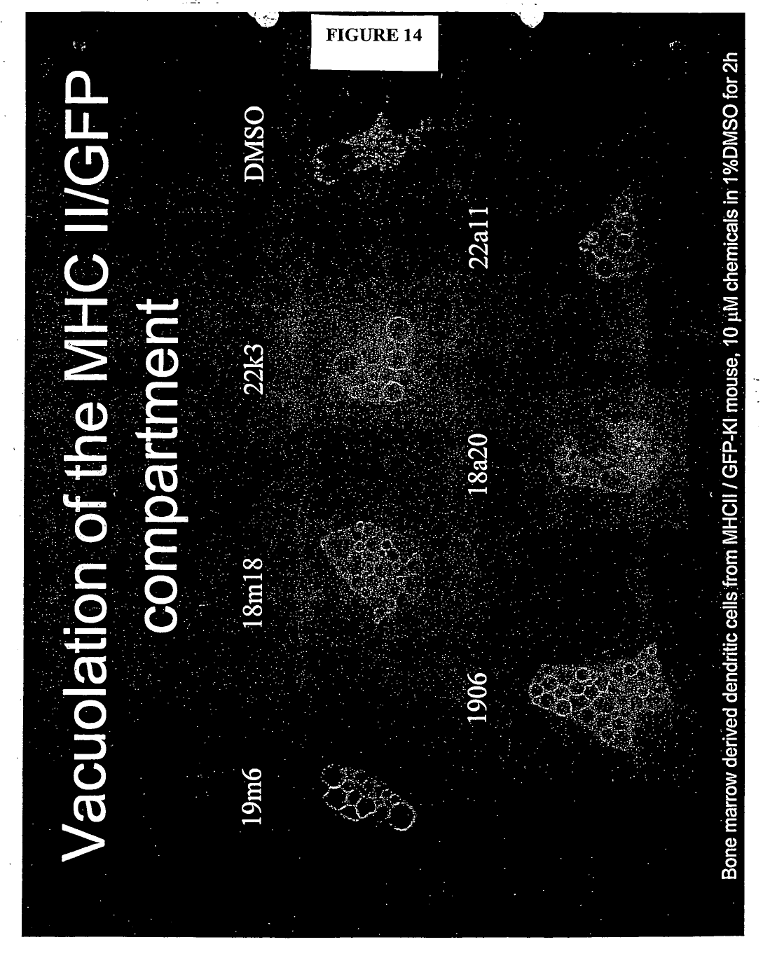

Exposure of cells in culture to inventive vacuolin compounds blocks cell division but, as shown in FIG. 13, does not kill the cells, which still endocytose transferrin by receptor-mediated endocytosis through the clathrin pathway.

### Example 3

### Vacuolins Block Induced Presentation of MHC Class II Antigen

Under normal conditions, most of the MHC class II molecules in dendritic cells are located in the cells, with a large fraction in the inner membranes of the multivesicular bodies. When such cells are stimulated with LPS, for example, or when they are first loaded with appropriate peptides and then exposed to T-cells carrying matching T-cell receptors, there is a rapid and massive movement of MHC class II molecules from the multivesicular compartments to the cell surface. This movement of MHC class II molecules necessarily also results in transport of antigenic peptides displayed within the MHC class II molecules, therefore resulting in a huge increase in antigen presentation.

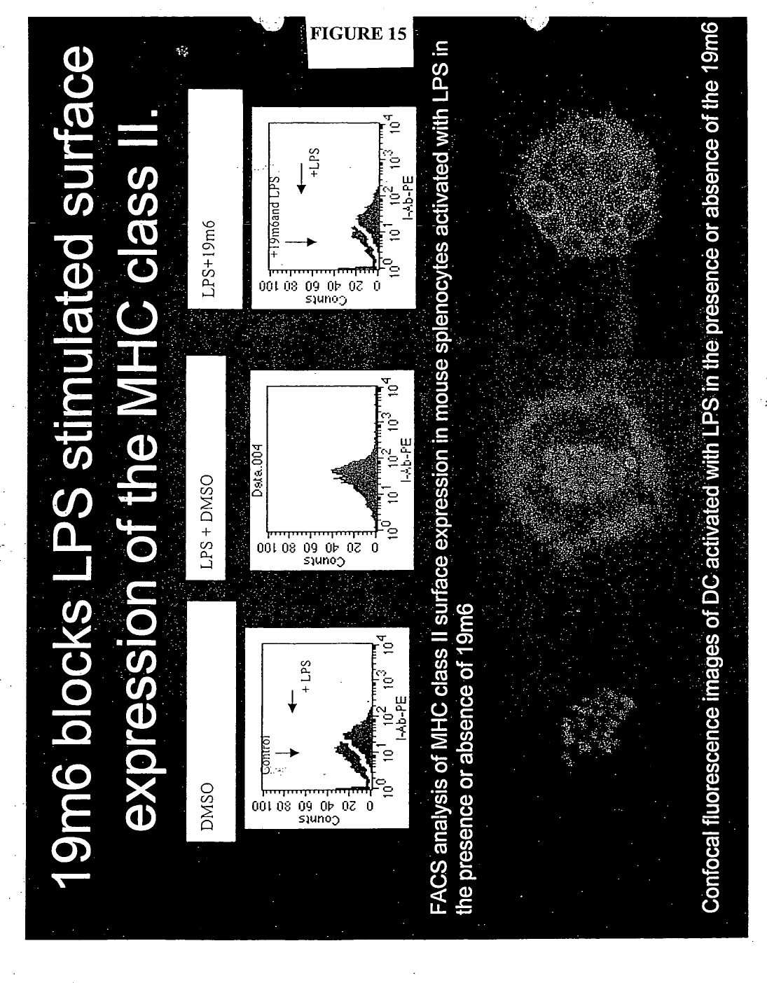

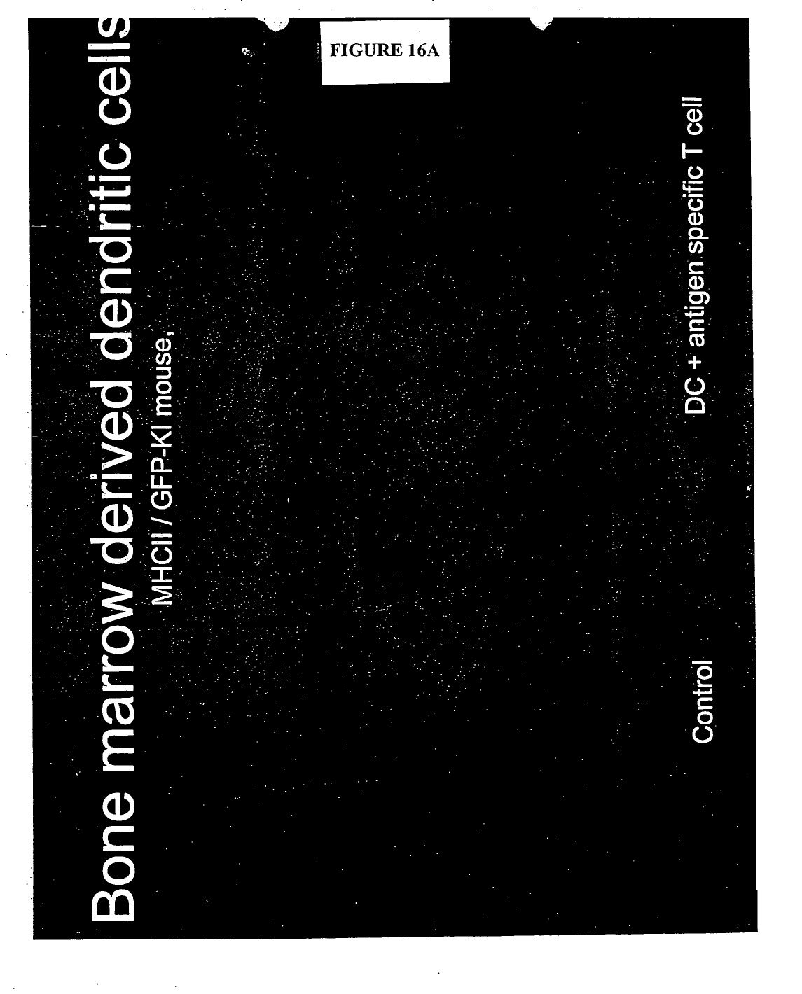

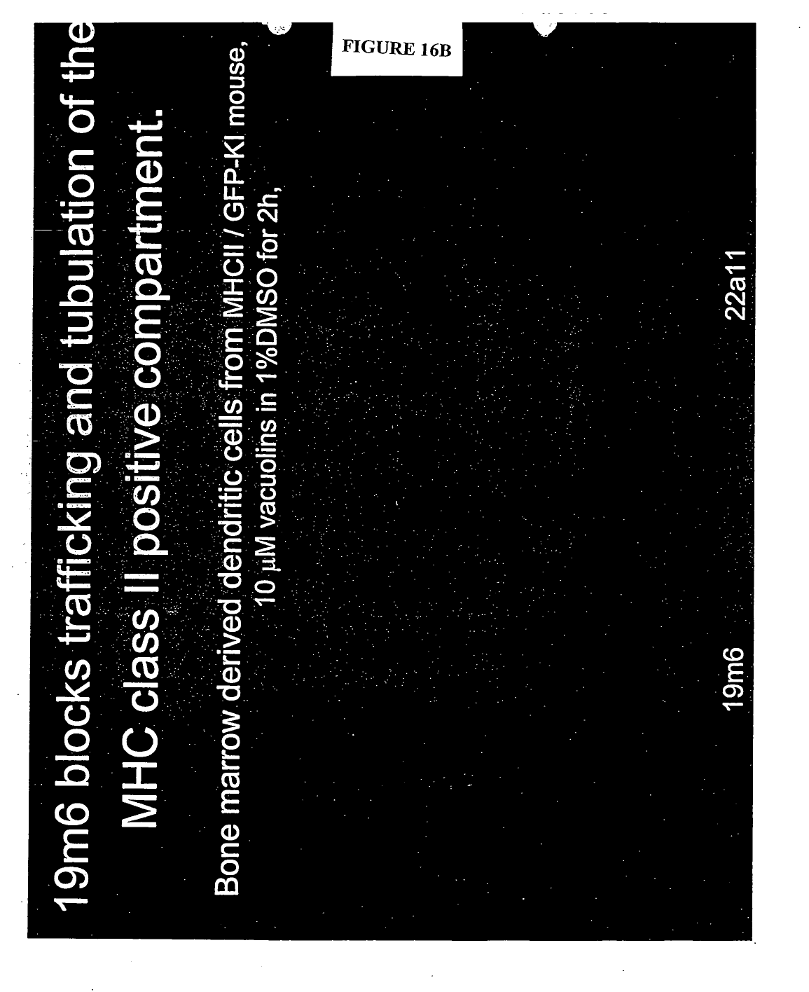

As shown in FIG. 14, inventive vacuolin compounds induce vacuolation of the MHC II/GFP compartment. The effects of these compounds on induced MHC class II movement were tested using mouse cells containing a knock-in MHC class II/GFP fusion. FIG. 15 presents representative data demonstrating that inventive vacuolins can block the LPS-stimulated surface expression of MHC class II molecules. Similarly, FIG. 16, panels A and B, show that inventive vacuolin compounds block trafficking and tubulation of intracellular compartments containing MHC class II molecules in GFP-KI murine dendritic cells.

These results demonstrate that inventive vacuolin compounds, and compositions containing them, can inhibit or prevent induced transport of MHC class II molecules, and therefore can block or reduce antigen presentation.

### Example 4

### Inventive Vacuolins Block Regulated Exocytosis of Lysosomes

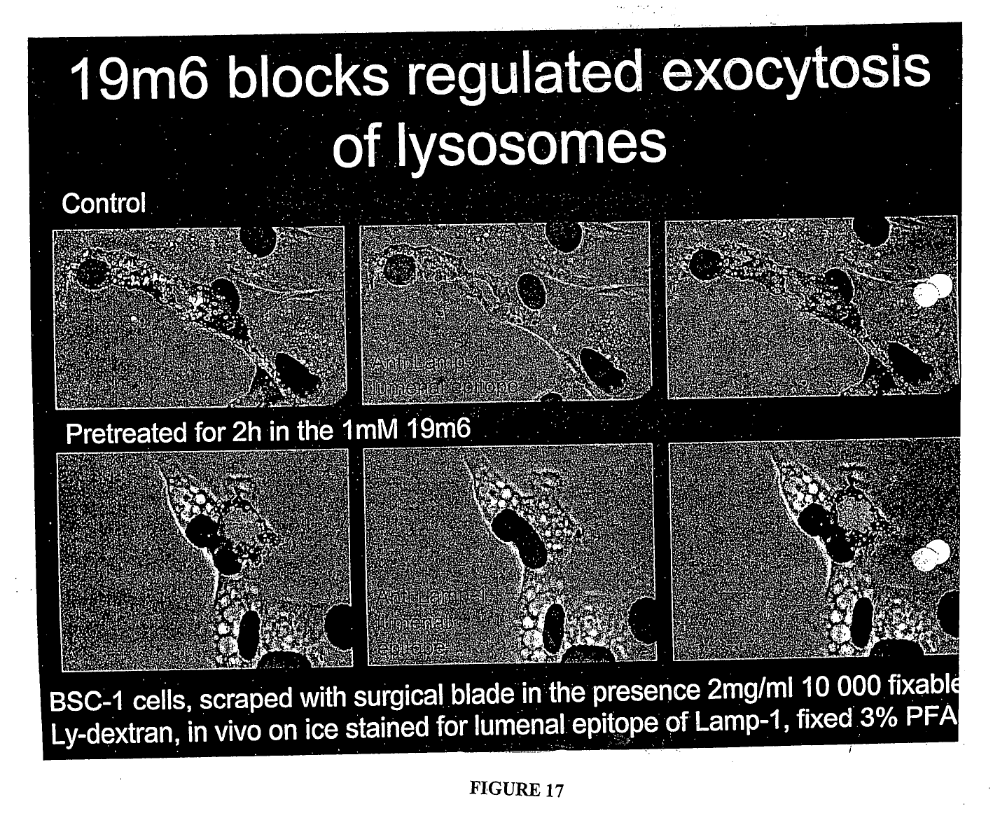

When cells in tissue culture are wounded, for example by scraping a part of the monolayer, they respond rapidly by inducing a healing response that includes the fusion of a portion of the mitochondrial membrane with the plasma membrane, resulting in the appearance of lysosomal markers on the cell surface. As shown in FIG. 17, inventive vacuolin compounds can block the appearance of lysosomal markers on the cell surface after scraping.

### Other Embodiments

Those of ordinary skill in the art will readily appreciate that the foregoing represents merely a description of certain preferred embodiments of the invention, and that the scope of the invention is not intended to be limited to these particular described embodiments; various modifications and substitutions will be apparent and are encompassed by the invention, as described in the appended claims.

1. A composition comprising a vacuolin or a vacuolin having the following structure: 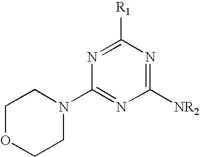
wherein R1 is an amino cycloaliphatic or cycloheteroalkyl group:

2. The composition of claim 1, wherein R1 is an aminoaryl group.

3. The composition of claim 1, wherein R1 is selected from the group consisting of a morpholino, a phenyl amine, a diphenylamine, a substituted phenylamine, a substituted diphenylamine, a benzylamine, and a substituted benzylamine.

4. The composition of claim 2, wherein the substituted phenylamine is halophenylamine selected from the group consisting of a chlorophenylamine, bromophenylamine, fluorophenylamine, and a iodophenyl amine.

5. The composition of claim 2, wherein the phenylamine has a para, ortho or meta substitution.

6. The composition of claim 1, wherein R2 is an arylakyleneamine group wherein R2 is an arylmethyleneamine, an arylethyleneamine, or an arylpropyleneamine group.

7. The composition of claim 1, wherein R2 is a phenylalkyleneamine group.

8. The composition of claim 1, wherein R2 is a benzylideneamine group or a benzene.

9. The composition of claim 1, wherein R2 is substituted with a halogen selected from the group consisting of fluorine, bromine, chlorine, and iodine.

10. The composition of claim 1, wherein R2 has a meta, para or ortho substitution.

11. A composition comprising a vacuolin selected from the group consisting of at least one of the vacuolin structures shown in FIG. 1.

12. A method of treating an intracellular trafficking-related disease or disorder in a subject, comprising administering to a subject an effective amount of the composition of claim 1.

13. A pharmaceutical composition comprising an effective amount of the composition of claim 1.

14. A method for modulating vacuolarization in a cell comprising:
providing a cell; and
contacting said cell with an effective amount of the composition of claim 13, thereby modulating vacuolarization in a cell.

15. A method of inhibiting antigen presentation mediated by MHC class II molecules, comprising:
providing a population of target cells containing internal MHC class II molecules; and
contacting the target cells with an effective amount of the composition of claim 13, so that, if the cells were triggered to externalize the MHC class II molecules, such externalization would be reduced as compared with externalization by comparable cells not so contacted.

16. A method of inhibiting externalization of intracellular compartments, the method comprising steps of:
providing a population of target cells containing intracellular compartments susceptible of externalization; and
contacting the target cells with an effective amount of the composition of claim 13 so that, if the contacted cells were triggered to externalize the intracellular compartments, a lower level of externalization would be observed than is seen with comparable cells not so contacted.

17. The method of claim 16, wherein the intracellular compartments contain MHC class II molecules.

18. The method of claim 17, wherein the step of providing comprises providing a population of graft cells containing MHC class II molecules that carry host protein fragments as antigens for presentation.

19. A method of inhibiting trafficking of intracellular compartments in a cell, the method comprising steps of:
providing a cell; and
contacting the cell with an effective amount of the composition of claim 13, thereby inhibiting trafficking of intracellular compartments.

20. The method of claim 19, wherein said cell is selected from the group consisting of: fibroblasts, macrophages, dendritic cells, and epithelial cells.
